(19) United States
(12) Patent Application Publication
(10) Pub. No.: US 2012/0289979 A1
(43) Pub. Date: Nov. 15, 2012

(54) Apparatus for Supplying Surgical Staple Line Reinforcement

(76) Inventors: Sherif Eskaros, Elkton, MD (US); Glenn P. Donahey, JR., Wilmington, DE (US); Matthew E. Doyle, Newark, DE (US); Conor R. McCrea, Landenberg, PA (US); Michael S. Winterling, Landenberg, PA (US)

(21) Appl. No.: 13/370,403

(22) Filed: Feb. 10, 2012

Related U.S. Application Data
(63) Continuation-in-part of application No. 12/247,710, filed on Oct. 8, 2008.
(60) Provisional application No. 60/978,178, filed on Oct. 8, 2007.

(51) Int. Cl. A61B 17/03

(52) U.S. Cl. 606/151

(57) ABSTRACT

An apparatus for supplying surgical buttress material to a surgical stapler is provided. The apparatus has a pivotable area for attaching surgical buttress material. The apparatus may also have an adhesive and a release liner disposed over the buttress material.

### CROSS REFERENCE TO RELATED APPLICATION

This application is a continuation-in-part of U.S. application Ser. No. 12/247,710, filed Oct. 8, 2008, which claims priority to U.S. Provisional Application No. 60/978,178, filed Oct. 8, 2007.

### FIELD OF THE INVENTION

The present invention relates to an apparatus for supply of surgical staple line reinforcement material to a surgical stapler.

### BACKGROUND OF THE INVENTION

Surgical stapling devices are employed by surgeons to sequentially or simultaneously apply one or more rows of surgical fasteners such as staples or two-part fasteners to body tissue for the purpose of joining segments of body tissue together. Such stapling devices generally consist of a pair of jaws (one being the cartridge side and one being the anvil side) or arm-like structures between which body tissue to be joined is placed. When the instrument is actuated, or “fired,” longitudinally moving firing bars contact staple drive members in one of the jaws, thereby pushing surgical staples through body tissue and into an anvil in the opposite jaw which crimps the staples closed. If tissue is to be removed, a knife blade can be provided to cut between lines of staples. Examples of such instruments are described in U.S. Pat. Nos. 4,354,628, 5,014,899 and 5,040,715, teachings of each of which are incorporated herein by reference.

Surgical stapling devices have found widespread application in surgical operations where body tissue must be joined or removed, such as, bariatric, colorectal, and thoracic procedures. When operating on thin tissue, such as thin emphysematous lung tissue, it is important to effectively seal the tissue which can be particularly prone to air leakage. Preventing or reducing air leakage can significantly decrease post operative recovery time. Inhibiting or preventing tearing at the stapled incision site in musculoaponeurotic areas also significantly decreases recovery time.

Thus, structures for use with surgical stapling devices which enhance sealing at the surgical site and/or reduce tissue tearing are desired.

U.S. Pat. Nos. 5,503,638, 5,575,803, 5,549,628, 5,702,409, 5,810,855 and 5,908,427 describe surgical staple line reinforcement devices and surgical stapling apparatus with tissue bolstering materials.

U.S. Pat. No. 5,752,965 and U.S. Pat. No. 6,939,358 describe apparatus for applying surgical reinforcement material onto the working surface of a surgical stapler without requiring removal of excess material.

The present invention provides a long felt need for an apparatus that quickly and easily applies a thin and flexible surgical reinforcement material to a stapler.

### SUMMARY OF THE INVENTION

The present invention is an improved apparatus for quickly and easily applying a thin and flexible surgical reinforcement material to a surgical stapler. The present apparatus comprises a body having a surgical reinforcement material attached to a portion of said body, the portion of the said body having the reinforcement material thereon being pivotable with respect to the remainder of said body. A touch-up pad may be attached to the apparatus, or otherwise provided as part of a kit. The apparatus further comprises a securement for temporarily fixing a surgical reinforcement material to the apparatus. The body of the apparatus may be configured so that orientation of the apparatus body with respect to a surgical stapler is able to be reversed with respect to the first side and the second side of the apparatus body.

The surgical reinforcement material may further comprise an adhesive on the outermost surface of the surgical reinforcement material with respect to said body. The adhesive may be any suitable adhesive including but not limited to a pressure sensitive adhesive.

### BRIEF DESCRIPTION OF THE DRAWINGS

FIG. 1 is a drawing of an embodiment of an apparatus of the present invention.

FIG. 2 is a drawing of an embodiment of an apparatus of the present invention with a hinged body.

FIG. 3A is a drawing of an embodiment of a reversible apparatus of the present invention exhibiting securements in the form of posts.

FIG. 3B is a drawing of an embodiment of an apparatus of the present invention exhibiting securements in the form of posts and an optional touch-up pad.

FIG. 4 is a drawing of an embodiment of an apparatus of the present invention showing an optional touch-up pad attached to the body via a connecting means.

FIGS. 5A through 5D are drawings showing different embodiments of touch up pads useful in the present invention.

FIG. 6 A through 6E are illustrations of the apparatus of the present invention with the pivoting portion of the apparatus shown bending out of the plane of the card.

FIG. 7 is a perspective view of an embodiment of an apparatus for applying a buttress or surgical material.

FIG. 8A is a perspective view of an embodiment of an apparatus for applying a buttress or surgical reinforcement material.

FIG. 8B is a perspective view of a base member of the apparatus shown in FIG. 8A.

FIG. 8C is a perspective view of the buttress or surgical reinforcement material of the apparatus shown in FIG. 8A.

FIG. 9A is a perspective view of an embodiment of an apparatus for applying a buttress or surgical reinforcement material.

FIG. 9B is a perspective view of a base member of the apparatus shown in FIG. 9A.

FIG. 10A is a drawing of an embodiment of an apparatus for applying a buttress or surgical reinforcement material.

FIG. 10B is a drawing of a base member of the apparatus shown in FIG. 10A.

FIG. 10C is an end view of an apparatus of the present invention with surgical buttress material disposed thereon.

FIG. 11A is a drawing of an embodiment of an apparatus for applying a buttress or surgical reinforcement material in an unconstrained condition.

FIG. 11B is a drawing of an embodiment of an apparatus for applying a buttress or surgical reinforcement material in a constrained condition.

FIG. 12A is a drawing of an embodiment of an apparatus for applying a buttress or surgical reinforcement material.

FIG. 12B is a drawing of a base member of the apparatus shown in FIG. 12A.

FIG. 12C is a drawing of the buttress or surgical reinforcement material of the apparatus shown in FIG. 12A.

FIG. 12D is a drawing of the elastic component of the apparatus shown in FIG. 12A.

FIG. 13A-E is a series of perspective and end views of one embodiment of an apparatus for applying a buttress or surgical reinforcement material in combination with a surgical stapler cartridge reload device.

### DETAILED DESCRIPTION OF THE INVENTION

The present invention provides an apparatus for supplying surgical reinforcement material to a surgical stapler.

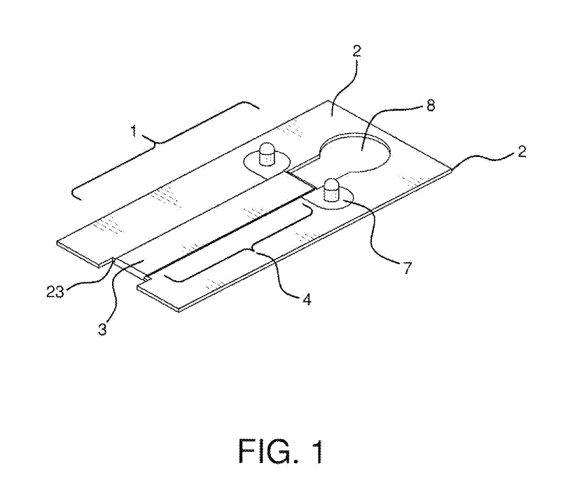

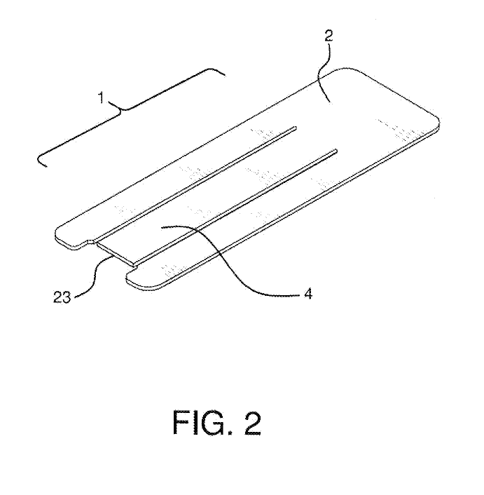

As shown in FIGS. 1 through 6, the apparatus 1 of the present invention comprises a body 2 sized to provide a surgical stapler with surgical reinforcement material (FIG. 1), on at least a portion of the open stapler arms of the surgical stapler.

FIG. 1 shows a schematic of the apparatus comprising a body having a first side (illustrated in this Figure as oriented on the top of the apparatus) and a second side. The body 2 of the apparatus also has a first portion and a second portion. The first portion 4 of the body is pivotable with respect to the second portion of the body. For clarity of the invention it is to be understood that the pivotable portion of the body as hereinafter described, is considered to be the first portion 4 of the body of the apparatus. A surgical reinforcement material, hereinafter referred to as “buttress material” is further disposed over an area of the first portion 4 of said body. In one embodiment the buttress material 3 is secured to the second portion of the body. The body is be sized appropriately to receive a surgical stapler in a manner that permits application of the buttress material 3 onto the jaws of the stapler.

The body has a first side 40 and a second side 50 that may be interchangeable for ease of loading a surgical stapler with buttress material. While FIG. 1 illustrates the pivotable first portion 4 of the body 2 as being located in a relative central position with respect to the remainder of the body 2, it is understood that the pivotable portion 4 may be located in other desired positions. The pivotable first portion 4 of the body functions to break the buttress material 3 at perforated areas (such as perforated tabs) so that the buttress material is easily freed from securements 7 on the apparatus. For example, the pivoting first portion bends out of the plane of the apparatus in at least one direction to separate the buttress material 3 at designated areas to prepare for application. The pivoting portion (first portion 4 of the body) of the apparatus is shown as a rectangular shaped portion, however, it is to be understood that other suitable shapes such as rods, loops, wires, rigid meshes, thin strip members, or other shapes may be desired. FIG. 1 also illustrates the body formed with an open central region 8. The open central region 8 may be incorporated into the body for ease of movement of the stapler with respect to the body.

The buttress material 3 is in contact with at least an area of the pivotable portion 4 of said body 2. Buttress material 3 suitable for use with the present invention can be fabricated from any material having acceptable biocompatibility and mechanical properties. For instance, the buttress material 3 may be formed of any suitable material and may be either synthetic or animal-derived. While synthetic materials are preferred due to, for example, their ability to be formed thinly as described herein and for other manufacturing benefits, animal-derived buttress materials may also be used. The buttress material 3 may be a single piece which is wrapped around the body of the apparatus, as shown in FIG. 1, or may be multiple pieces.

Animal-derived buttress materials (such as, for example, bovine, porcine, equine, and human products) may have difficulties associated with their placement on the working surfaces (for example the anvil and cartridge jaw surfaces) of surgical staplers. Animal-derived buttress materials cannot typically reach the consistent and thin profiles while maintaining the required strength associated with preferred synthetic buttress materials. An advantage of the present invention is that unlike traditional applications cards which do not allow successful placement of thin buttress materials, a buttress material 3 having a thickness less than 0.5 millimeters may be accurately and successfully positioned onto the working surfaces of a surgical stapler using the present apparatus.

Further, animal-derived buttress materials routinely use added steps to apply or actuate adhesives to the outer surface of their materials. For instance, some products utilize rehydration techniques, while others utilize paste-like or gel-type adhesives which need to be applied and smoothed onto the buttress material 3 prior to use. Paste-like or gel-type adhesives allow for sliding of the buttress materials on the working surfaces of a surgical stapler during loading. The present invention does not require added application or actuation steps when a ready to use adhesive such as a pressure sensitive adhesive is employed. A pressure sensitive adhesive can be ready to use by simple removal of a release liner.

An unexpected advantage of the present apparatus is that thinner buttress materials such as synthetic non-animal derived materials and other materials particularly those less than 0.5 millimeters in thickness are more readily applied to a stapler working surfaces than is possible with traditional applicator means. The buttress material 3 is able to be readily adhered or placed back to the crotch of a surgical stapler and successfully cover the staple lines with the present apparatus, because the body can be made, thin enough to reach to the crotch of a stapler. This ease of placement also allows the buttress material 3 to be attached onto the working surfaces of a stapler without bunching.

Examples of suitable synthetic buttress materials include but are not limited to: bioabsorbable or biodegradable materials or webs, non-bioabsorbable materials or webs, expanded polytetrafluoroethylene (“ePTFE”) such as disclosed in U.S. Pat. No. 5,810,855, synthetic materials, or other suitable buttress materials. One advantage of using synthetic buttress materials is that there is no need to rehydrate synthetic materials prior to use, and they will have a more consistent thickness. The surgical buttress material 3 may further comprise an adhesive on at least the outermost surface relative to the body of the apparatus. The adhesive may be a ready to use adhesive such as a pressure sensitive adhesive or further may be an activatible adhesive which may be moistened or otherwise activated prior to use. A release liner, such as PET release films, or other coated webs or sheets may be applied over an adhesive to protect it until it is ready for use. The release liner may be utilized to cover the buttress material 3 and pre-applied adhesive so that the liner may readily separate from the adhesive on the surgical buttress material 3, thus maintaining the integrity of the adhesive, and allowing it to be easily prepared for use. One advantage of the present apparatus is that the adhesive may be pre-applied and essentially be uniformly distributed over the buttress material. Thus, eliminating the need for any smoothing or spreading as required by unevenly distributed gel or paste-like adhesives traditionally used on buttress materials (see Example 10).

The apparatus 1 is sized to apply surgical buttress material 3 onto a surgical stapler. The apparatus may comprise the same material throughout the body or may comprise two or more different materials, such as, but not limited to plastics, metals, cardboards, foams, or any other suitable materials.

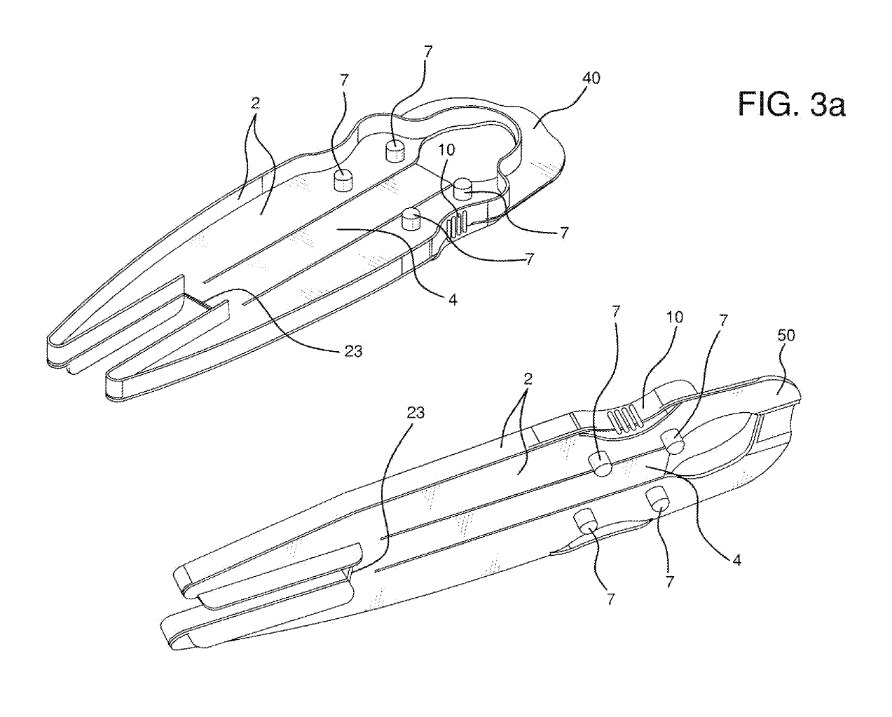

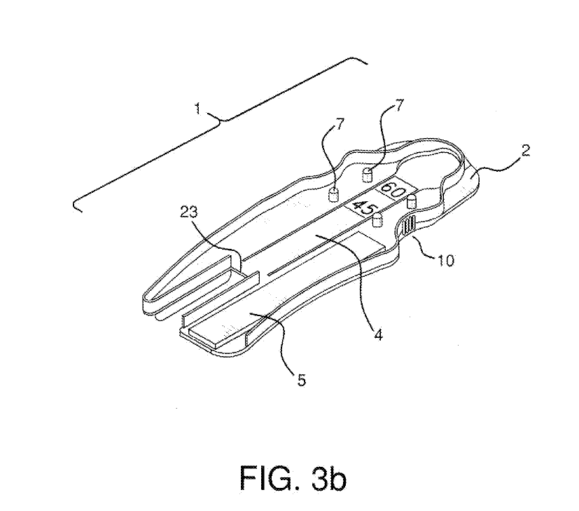

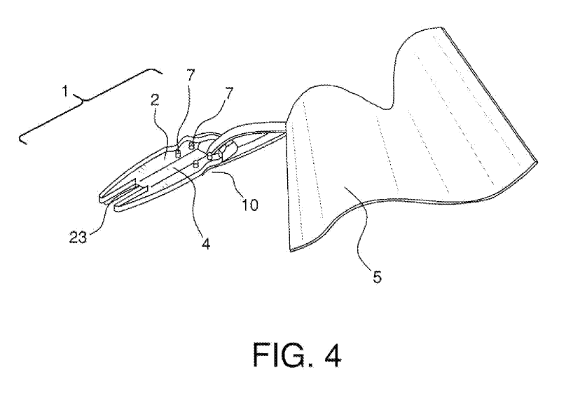

The apparatus 1 further comprises one or more securements 7 for temporarily fixing buttress material 3 to the apparatus 1. Buttress material 3 is temporarily affixed to the apparatus via this securement to allow the buttress material 3 to extend to about at least a section of the first portion 4 of the body. In one embodiment, as depicted in FIG. 1, one securement is simply an affixed tab with a perforated section formed of additional buttress material 3. The optional post is shown as additional securement means. The securements 7 are further illustrated in FIGS. 3A, 3B, and 4 as comprising a plurality of posts on each body positioned parallel to each other near the pivotable first portion 4 of the body. The posts may be designed to provide tension on the buttress material 3 so that the buttress material 3 is stretched flat, with reduced bunching or wrinkling and provide a more even contact surface to adhere onto a stapler arm. It is to be understood that the posts can be positioned non-parallel to each other in alternate embodiments. These posts are sized and positioned on the bodies to fit perforations in the surgical buttress material 3. Other forms of securement 7 include tabs with adhesive bonding, frictional interference, ultrasonic or thermal welding, mechanical attachments, or any other suitable means of securing the buttress material to the body.

As shown in FIGS. 3A, 3B, 4, 5A-D, and 6 A-6 E, the apparatus of the present invention may further comprise a touch-up pad 5. FIG. 4 illustrates a touch-up pad 5 which may be permanently or removably attached to the apparatus as an interconnected element.

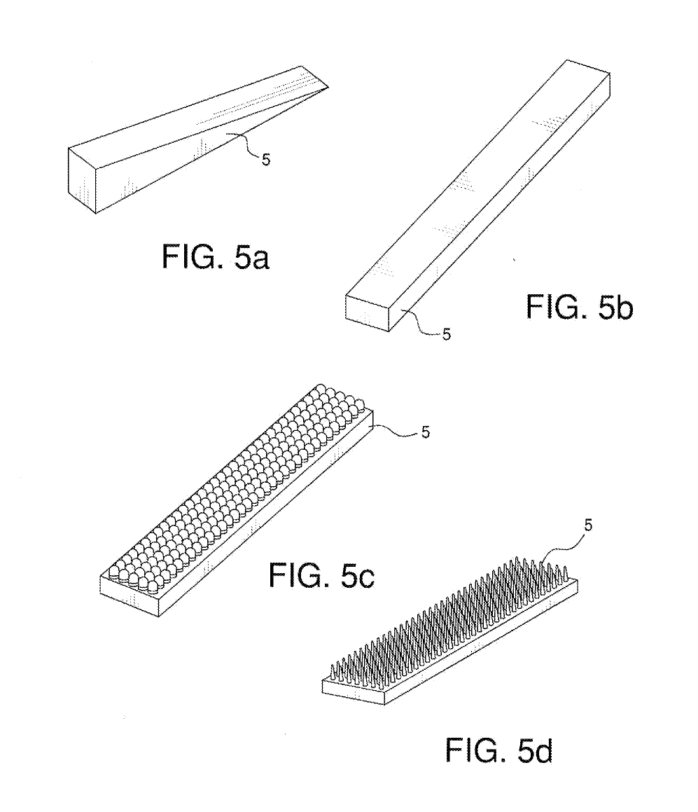

Alternatively, as shown in FIG. 3A shows an embodiment of the present apparatus which allows the body of the apparatus to be reversibly inserted into a surgical stapler and perform in either inserted configuration. The reversible embodiment of the apparatus comprises a body having a first side 40 and a second side 50 which may be fed into a stapler irrespective of which side is in contact with the anvil and which side is in contact with the cartridge of the stapler. The reversible configuration enables a practitioner the ability to easily load the body into the stapler. Also shown in FIGS. 3B and 5, an optional touch-up pad 5 may be permanently or non-permanently attached to the body of the apparatus on one or both sides. The touch-up pad is used to maximize the surface area contact between the buttress material 3 and the working surface of the stapler. For instance, adhesive 6 on the outermost facing portion of buttress material 3 adheres the buttress material 3 onto opposed working surfaces of a surgical stapler. After the buttress material is oriented onto the apparatus, it is sometimes desired that the buttress material be further pressed onto a touch pad to adhere the buttress material onto a larger surface area of the stapler. This is especially advantageous when the adhesive on the apparatus is a pressure sensitive adhesive disposed over the surgical buttress material.

As shown in FIGS. 5A through 5D, the touch-up pad 5 may be of a uniform or non-uniform topography, further the touch-up pad may comprise of one or multiple pieces, it may be designed as a wedge (for example, FIG. 5A), flat pad (for example, FIG. 5B), raised dots (for example, FIG. 5C), or the like. It is sometimes desirable that the touch-up pad have specific formations targeted to enhance the surface area around the staple line. The touch-up pad is generally a material such as, but not limited to cottons, polyesters, foams, plastics, metals, or any other suitable materials. It is an advantage to have the touch up pad located in an area apart from the buttress material 3 so that no added thickness or bulk is associated with the buttress material, or apparatus, during application of the material to the arms of the stapler.

In the embodiment shown in FIGS. 3, 4 and 6A through 6E, the body of the apparatus 1 is shaped to include a finger grip 10 to facilitate gripping of the apparatus during loading of a surgical stapler with surgical buttress material 3. The end of the apparatus proximal to the pivotable first portion 4 of the body further comprises a stapler restraining area 23. The stapler restraining area 23 defines the origin or the receiving area for the buttress material (not shown) on the pivotable portion 4 of the body. The created pivotable portion is formed via the restraining area 23 which is in contact with the crotch of a stapler which allows pivoting of the receiving area of the first portion of the body, with respect to the plane of the second portion of the body, to affix buttress material to a stapler.

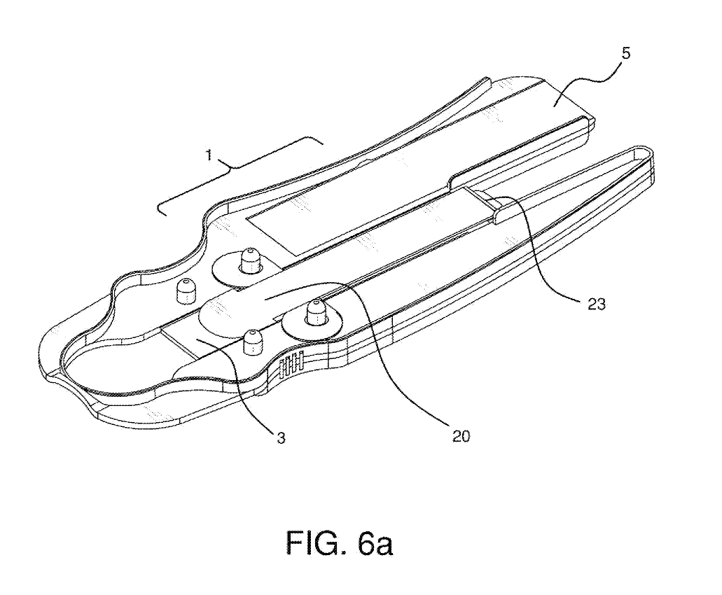

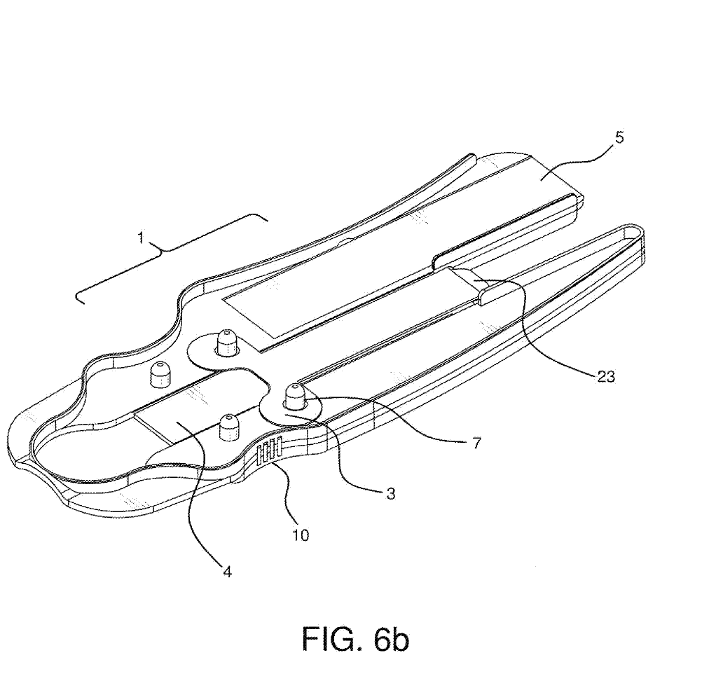

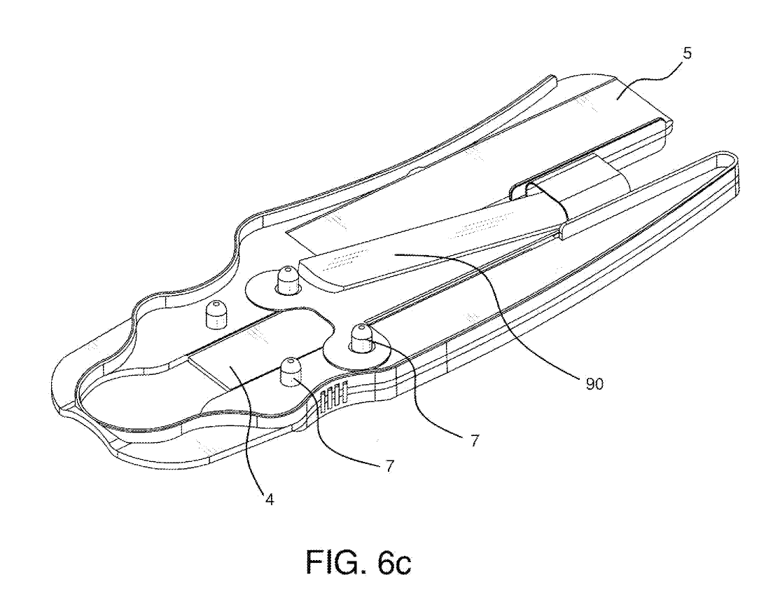

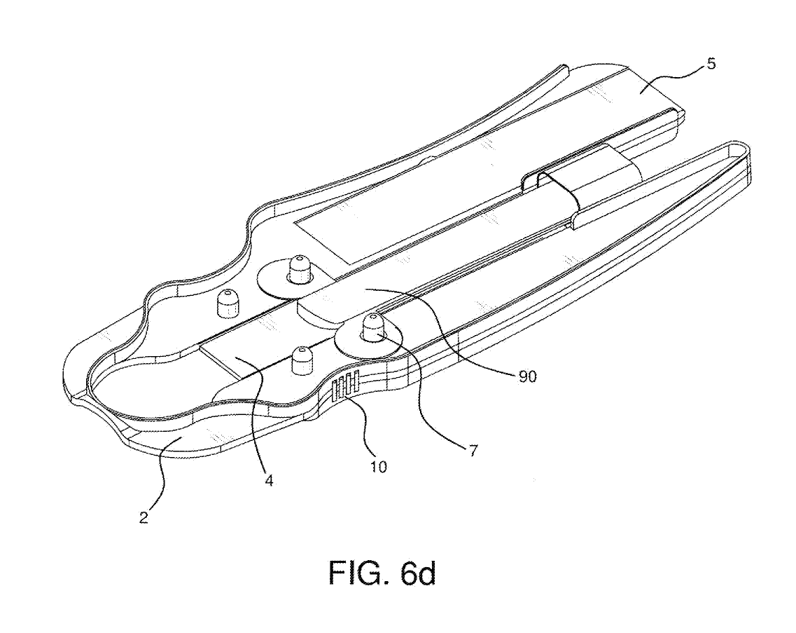

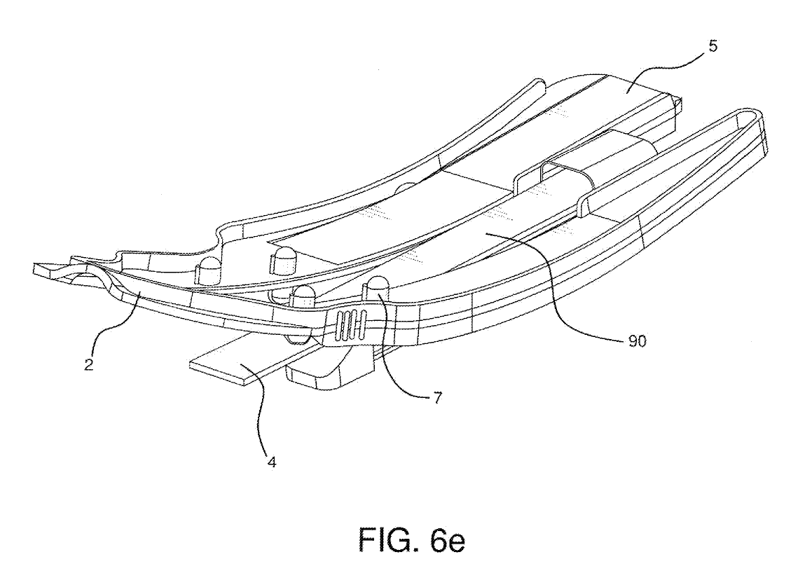

As shown in FIGS. 1 and 6A through 6E, the buttress material 3 is oriented on the body of the apparatus in order to allow a surgical stapler to clamp onto the apparatus. The adhesive 6 on the buttress material 3 adheres the buttress material 3 onto the opposed working surfaces of the surgical stapler. It is advantageous when the adhesive on the apparatus is a pressure sensitive adhesive disposed over the surgical buttress material. Prior to use, a release liner 20 may be utilized to cover the pressure sensitive adhesive and keep the adhesive in a ready to use form for application. Removal of the release liner exposes the pressure sensitive adhesive, thus enabling the practitioner to easily and quickly position the buttress material 3 onto the working surface of a surgical stapler. FIG. 6A shows a loaded apparatus 1 with a release liner 20 disposed over the buttress material 3. FIG. 6B shows a loaded apparatus 1 where the release liner is removed from the buttress material 3 exposing the adhesive on the buttress material and making it ready to engage with a stapler arm. FIG. 6C shows a stapler jaw 90 and the apparatus positioned so that the exposed adhesive on the buttress material will make contact with the working surface of the stapler jaw to adhere the buttress material onto the stapler. FIG. 6D shows a stapler jaw 90 clamped onto the apparatus 1 to allow contact between the exposed adhesive on the buttress material and the working surface of the stapler. FIG. 6E shows a the first portion 4 of the body pivoting out of the plane of the second portion of the body so that the stapler jaw 90 engaged with the first portion 4 moves to break the buttress material 3 at perforated areas and free the buttress material from securements 7 on the apparatus.

Other optional features can be added to the body including color coding of the body for ease of loading; measurement ticking, non-slip portions, or other desirable variations. Ergonomic features are also within the skill of one in the art to apply to the present apparatus such as, finger holds, curved surfaces, ribs or other raised features for additional tactile feel or grip ease especially with surgical gloves.

As described above, the apparatus of the present invention provides a simple method for applying buttress material 3 to a variety of staplers. The body of the apparatus is pre-loaded with buttress material 3 either by an artisan or as purchased in a ready to use embodiment. By “pre-loaded” it is meant that the buttress material 3 is attached to at least a portion of the body which is pivotable with respect to a second portion of said body. The release liner is removed from the buttress material, exposing the adhesive on the exposed surface of the buttress material. As shown in FIG. 6, the apparatus 1 is then inserted between open stapler arms and aligned with a stapler arm so that the buttress material 3 is in position to be applied to the arms of the stapler upon contact there between. The stapler arms are closed upon the pivotable portion of the body housing the buttress material, the pivotable region of the body is moved away from the plane of the remainder of the body so that the perforated tabs of the buttress material 3 are broken. If desired the touch-up pad may be utilized to maximize the surface area of the buttress material adhered to the stapler arms. The stapler arms are then opened to complete the transfer of the buttress material 3 to the stapler arms. The body of the apparatus is then removed from the stapler arms and discarded and the stapler is ready for use.

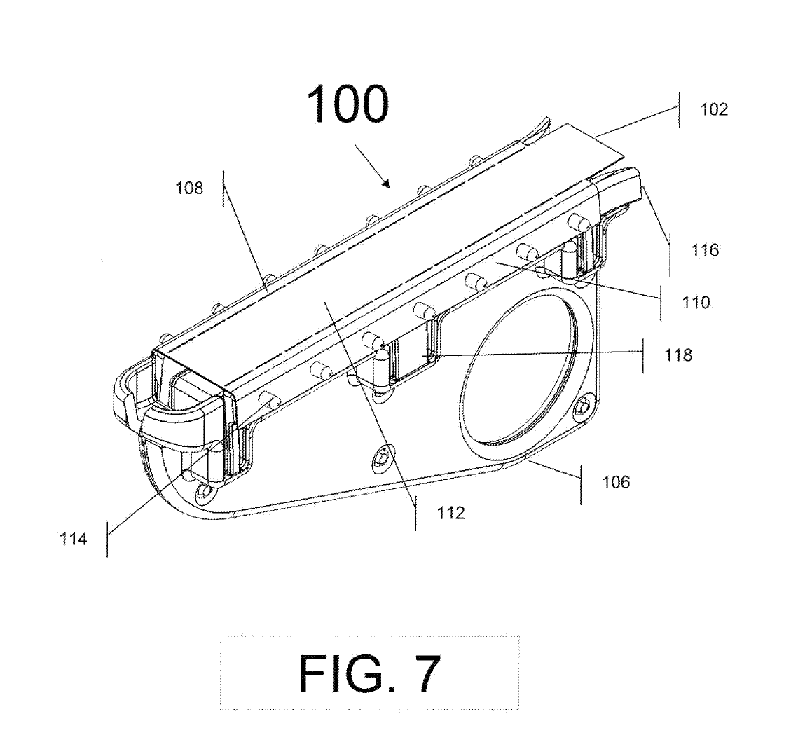

Turning now to FIG. 7A, another embodiment of an apparatus 100 for applying a buttress or surgical reinforcement material 102 to a stapler jaw is shown. FIG. 7B shows the apparatus 100 in combination with the jaws of a surgical stapler.

The apparatus 100 for applying a surgical reinforcement material may include a buttress material 102 having at least a portion 112 of buttress material that is releaesably coupled to a support or base member 106. The buttress material 102 may be secured to the base member by an adhesive or by a mechanical mechanism. The buttress material may include perforations 108 along the length of the buttress material 102. The mechanism used to secure the buttress material 102 to the base member 106 must be sufficient to maintain the coupling of the edges 110 of the buttress material to the base member 106 when sufficient force is applied to separate the center portion 112 of the buttress material from the edges 110 of the buttress material 102. In FIGS. 7A and 7B, the buttress material 102 is shown coupled to the base member 106 via posts 114 extending from the base member 106.

The base member 106 may include a pair of flexible tensioning arms 116. The posts 114 extend from the tensioning arms 116. The edges 110 of the buttress material include apertures 118 spaced to mate with the posts 114 extending from the tensioning arms 116. The apertures 118 are also spaced so that when the buttress material is coupled to both sets of posts 114 a tension force is applied to the tensioning arms 116. The tensioning arms 116 are designed to flex towards each other so that the buttress material 102 may be mounted to the base member 106. After the buttress material 102 is coupled to the attachment posts 114, the tensioning arms 116 are allowed to flex outwardly. The outward flex of the tensioning arms 116 creates tension in the buttress material 102 that is sufficient to hold the buttress material 102 flat. The base member 106 may also have hinges 118. The hinges 118 allow the base member 106 to bend and deform throughout storage while still holding the buttress material 102 in tension.

Base member 106 may further comprise an alignment mechanism to facilitate proper alignment of the buttress material to the stapler jaw and ensure all staple holes are ultimately covered. Such alignment mechanism may include a flexible tongue which facilitates depth and side to side positioning by fitting within the pivoting mechanism of the stapler jaw. Such tongue could be located on the base member at a point further distal to the end of the buttress material. Alternatively, a channel in the base member which facilitates depth and side to side positioning of the buttress material by insertion of the distal end of a surgical stapler into the channel could provide a suitable alignment mechanism. In yet another embodiment, the alignment mechanism could be in the form of a plunger which engages the features on the outside or back of the stapler jaw could likewise facilitate centering and resist twisting of the buttress material curing loading of the stapler.

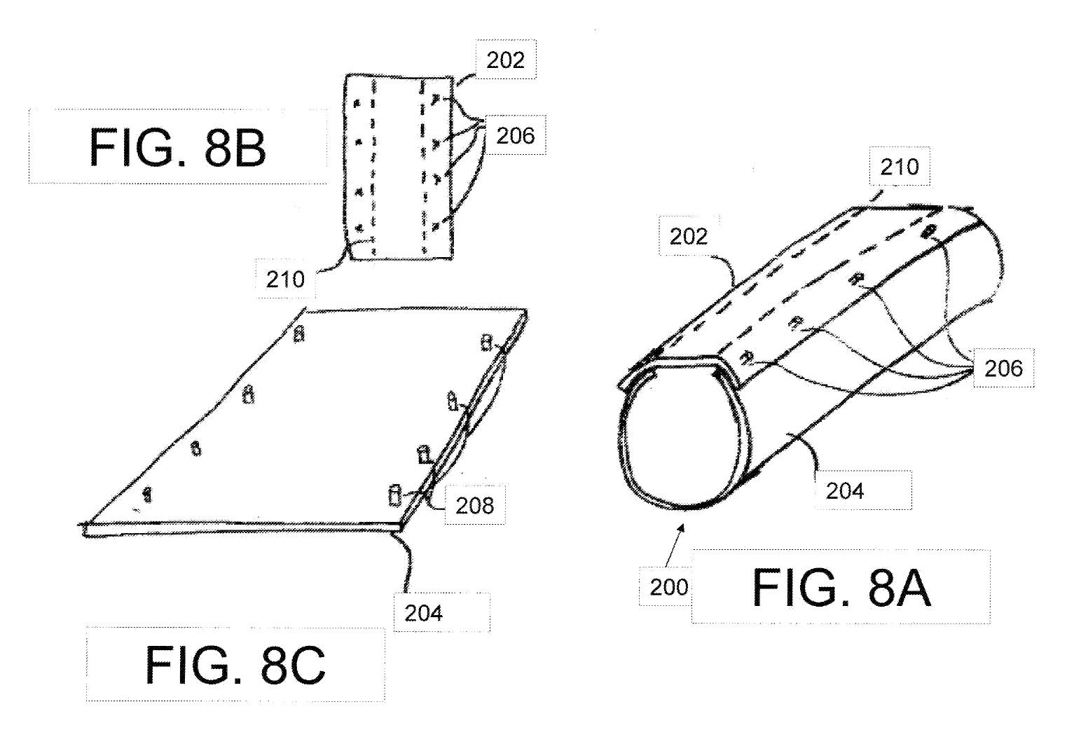

In FIGS. 8A-8C, an alternative embodiment of an apparatus 200 for applying a reinforcement or buttress material 202 is shown. The base member 204 may be made of a continuous sheet of semi rigid material. The base member 204 includes a coupling mechanism to secure a buttress material 202 to the base member 204. The buttress material 202 may have a plurality of apertures 206 spaced to align with a plurality of posts 208 on the base member 204. The buttress material may also include perforations 210 along the length of the device. The buttress material 202 is coupled to the base member 204 by bending the base member 204 into a C-shape and coupling the apertures 206 to the posts 208. The base member 204 must be rigid enough to hold the buttress material taut but not rigid enough to tear the perforations 210 prior to loading on the stapler jaw.

Suitable materials for the continuous sheet of semi rigid material or base member 204 include any number of thin plastic or metallic films. As illustrated in FIGS. 8A-8C, the buttress material 202 is shown secured by posts 208, any suitable mechanical or chemical bonding mechanisms may be used.

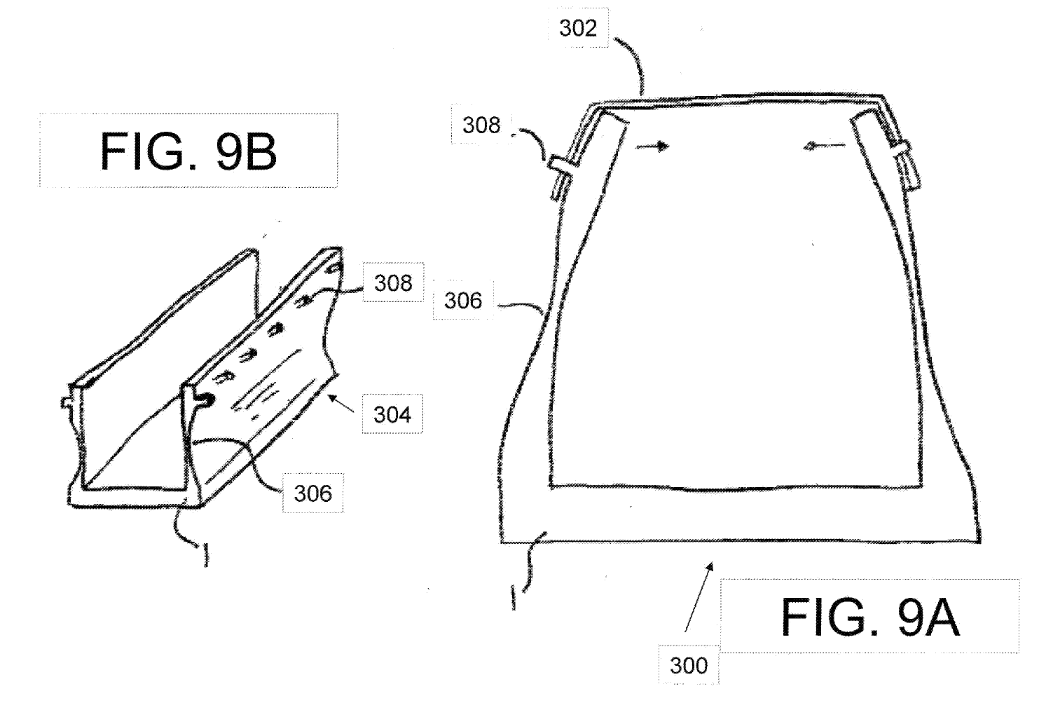

FIGS. 9A and 9B show an alternative embodiment of an apparatus 300 for applying a reinforcement or buttress material 302. The base member 304 may be an extruded piece of material specifically shaped to form a flexible hinge. The base member 304 may include at least a pair of tension or flex arms 306. In FIG. 9A, the flex arms 306 are shown in an unconstrained condition. The flex arms 306 may be squeezed together and placed in a constrained condition by a piece of buttress material 302 secured to the base member 304 as shown in FIG. 9B. The flex arms 306 will flex outwardly towards their unconstrained condition and provide a force to keep the buttress material 302 taut or in tension. The base member 304 is shown having posts 308 to secure the buttress material 302, however, any suitable retention mechanism may be used to hold the buttress material 302 in place.

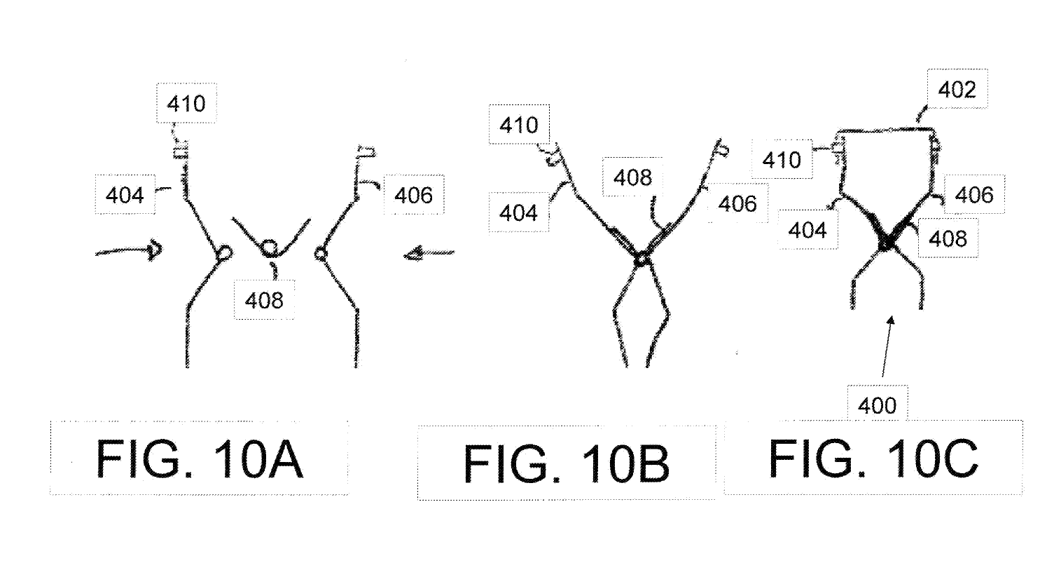

FIGS. 10A-10C show another alternative embodiment of an apparatus 400 for applying a reinforcement or buttress material 402 to a stapler jaw. The apparatus 400 has a first rigid arm 404 and a second rigid arm 406 coupled together with a spring member 408. FIG. 10B shows the apparatus 400 in an unconstrained condition before the buttress material 402 is mounted on the apparatus 400. The spring member 408 provides a force that biases the first and second arms 404, 406 into an open position as shown in FIG. 10B. The buttress material 402 is secured to the arms 404, 406 as shown in FIG. 10C. The distance between the coupling mechanisms (not shown) on each side of the buttress material 402 is less then the distance between the coupling mechanisms 410 on the rigid arms 404, 406 when the apparatus 400 is in an unconstrained state. The buttress material 402 may be stretched to couple with the coupling mechanisms 410 bringing the rigid arms 404, 406 closer together. The spring member 408 will provide a force that will push the rigid arms 404, 406 back towards an unconstrained condition. This force should be sufficient to keep the buttress material 402 in tension, but not enough to tear any perforations that may be provided in the buttress material.

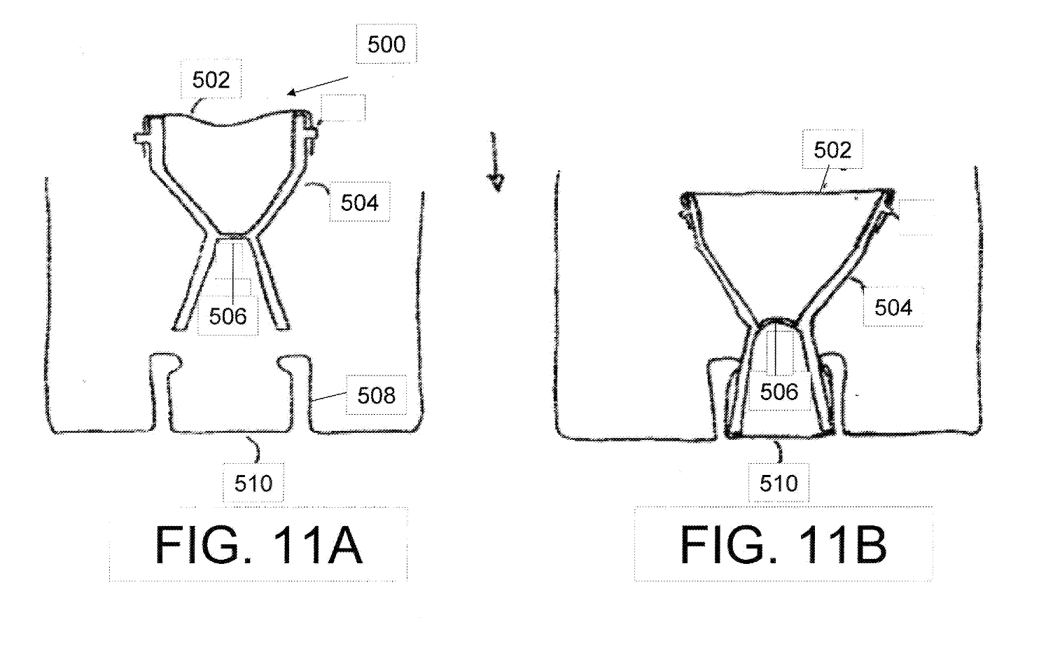

FIGS. 11A and 11B show an alternative embodiment of an apparatus 500 for applying a reinforcement or buttress material 502. Tension is applied to the buttress material upon storage of the apparatus 500 in a package 510. In FIG. 11A, the apparatus 500 is shown with a pair of flexible arms 504 coupled together through a flexible pivot joint 506. A pair of tension arms 508 is formed in a surface of the storage package 510. The tension arms 508 are spaced so that the arms 504 are placed in tension by the tension arms 508 of the package 510. The package 510 may be made from a variety of materials such as polyethylene terepthalate glycol (PETG) or high impact polystyrene (HIPS).

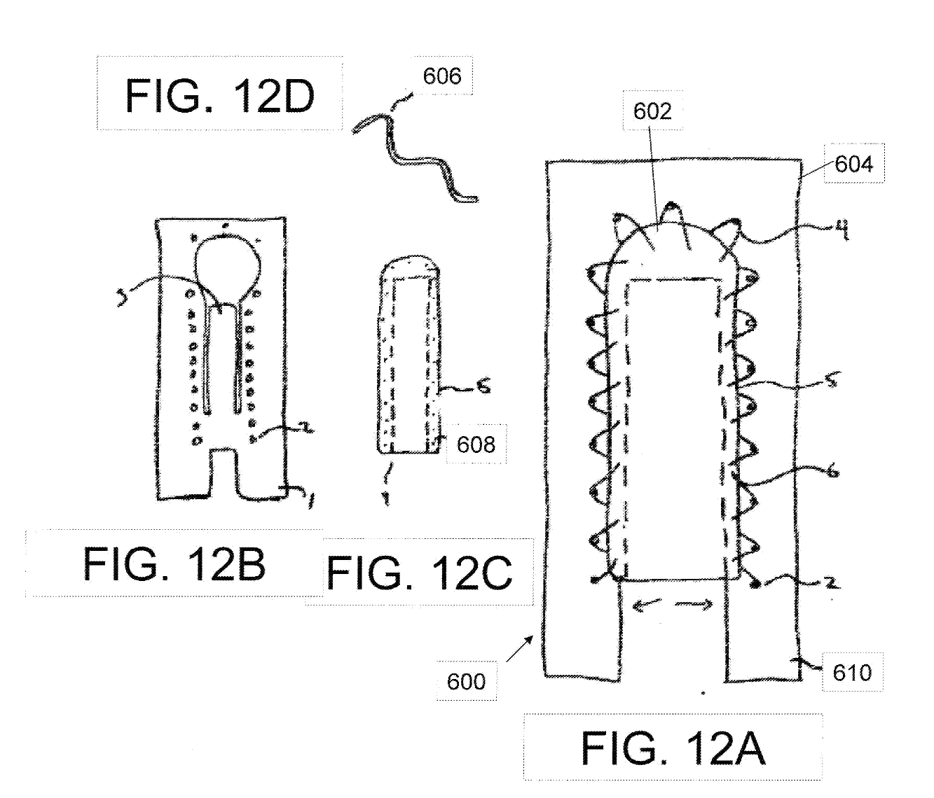

FIGS. 12A-12D show an alternative embodiment of an apparatus 600 for applying reinforcement or buttress material 602 to a stapler jaw. The applicator apparatus 600 may include a buttress material 602 secured to an applicator card 604 with an elastic component 606. The elastic component 606 couples the buttress material 602 to the applicator card 604 through features 608 in the buttress material 602 and features 610 provided on the applicator card 604. These features 608, 610 are shown as holes or apertures but may be any other feature that the elastic component 606 could be secured through such as posts or slits. The elastic component 606 is used to secure the buttress material 602 under tension so that any buttress material 602 is held taut when coupled to the applicator card. The buttress material 602 may be provided with perforations 612 so that when the apparatus is used to apply the buttress material 602 to a stapler only an amount needed to cover the surface of the stapler jaw is removed from the applicator card 604. The applicator card 604 may also include a pivoting portion 612. The pivoting portion 612 allows the surgeon to bend the pivoting portion 612 of the applicator card 604 when the surgeon is using the applicator card 604 to apply the buttress material 602.

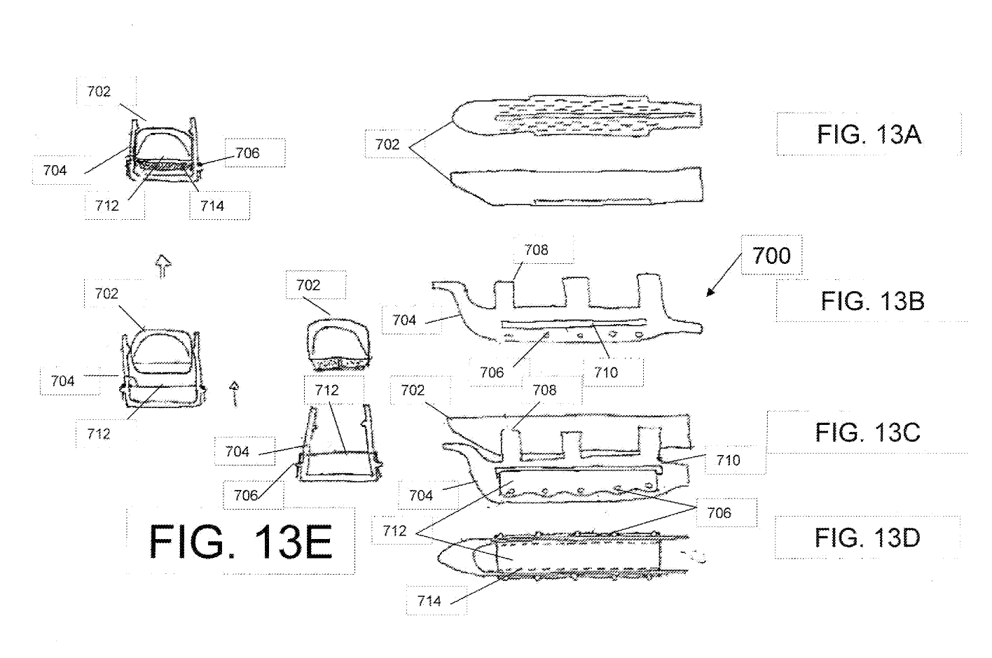

FIGS. 13A-E show another embodiment of an apparatus 704 for applying a reinforcement or buttress material 712 to a stapler jaw. The apparatus 704 is integrated into a cartridge reload 702. The apparatus is made of a flexible material with features designed to releasably couple at tabs 708 with the cartridge reload 702, and fix the buttress material 712 at pins 706 to enable delivery in tension. The buttress material 712 is fixed to the apparatus in an unconstrained state, in which case there is no tension in the system. When the apparatus 704 is combined with the cartridge reload 702, the system is placed in tension, thereby pulling the buttress material 712 taught and flat. The material can be perforated 714 to promote transfer from the delivery apparatus to the cartridge reload 702, upon delivery of the cartridge reload 702 to a surgical stapler.

### EXAMPLES

### Example 1

A 30 mil (0.076 cm) thick card forming the body of the apparatus (dimension 10 cm L×5.7 cm W) was made by injection molding polycarbonate and configured to aid in applying surgical staple line buttress (SLR) material to the jaws of a commercially available stapler. The SLR receiving area, stapler restraining area and the pivot of the SLR card were created through a three-sided cutout (S1×S2×S3) of the polycarbonate card using a razor tool. The lengths of the longitudinal cuts (S1 & S3) on the card are about ˜50% of the polycarbonate card (approximately 4.7 cm) and starting approximately 0.6 cm from one end of the card. The length of the latitudinal cut (S2) is sized to accommodate the width of the SLR and jaws of a commercially available stapler (approximately 1 cm).

The receiving area is defined by the three sides of the S1, S2 and S3 cuts. The uncut side of the receiving area defines the pivot. The end of the card closest to the pivot is the stapler restraining area. The restraining area defines the origin or the receiving area. The created pivot allows pivoting of the SLR receiving area with respect to the plane of the polycarbonate card.

### Example 2

One piece of SLR material with one side having a pressure sensitive adhesive (PSA) with a release liner for covering the PSA is placed on the receiving area of the polycarbonate card prepared as in Example 1.

The SLR material is precut with connected perforated tabs that extend beyond the width of the receiving area.

The SLR material is adapted to fit onto the receiving area and can be attached to the polycarbonate card on each side with perforated tabs that extend past the width of the receiving area. The perforated tabs of the SLR are attached to the polycarbonate card using a second PSA on the opposite side of the first PSA liner.

### Example 3

For a ready-to-use SLR card, the SLR is attached to the polycarbonate card prior to packaging.

### Example 4

After attaching the SLR to the polycarbonate card, the release liner covering the PSA is peeled back and removed. Next, the stapler is positioned against the restraining area of the card and then clamped over the polycarbonate card receiving area on the SLR with the PSA exposed adhesive. This securely attaches the stapler to the SLR. Next the card is moved perpendicularly to the stapler jaws at the opposing end of the restraining area to break the SLR from the perforated tabs. This releases the SLR from the card and allows the SLR to transfer completely to the stapler.

### Example 5

A 30 mil (0.076 cm) thick card (dimension 10.5 cm L×3 cm W) was made by injection molding polypropylene and configured to aid in applying SLR material to the jaws of a commercially available stapler. The injection molded card was designed to incorporate the SLR receiving area, stapler restraining area, and the pivot (as in example 1) in the mold design. Additionally, deformable posts were included in the design.

A receding section (dimension 2.7 cm L×1.2 cm W) is included in the design of the card such that the recession is adjacent to the restraining area on the opposite side of the receiving area. The receding section assists docking and positioning of the stapler jaws on the receiving area.

A precut SLR material (as in Example 2) with perforated tabs was adapted with holes to fit over the deformable posts. After positioning the SLR material onto the receiving area of the card, and positioning the perforated tabs over the deformable posts, the deformable posts were thermally deformed to capture the tabs and secure the SLR material to the card. Ultrasonic energy was used to thermally deform the posts.

Next the card is moved perpendicularly to the stapler jaws at the opposing end of the restraining area to break the SLR from the perforated tabs. This releases the SLR from the card and allows the SLR to transfer completely to the stapler.

### Example 6

A card with similar dimensions to Example 5 was created using stereolithography apparatus (SLA). The design of this card was modified to better adapt the card to a surgical stapler by creating a receiving area that is thicker at the distal end of the card than at the proximal end of the card. Additionally the receding section of the card was modified to allow the card and precut SLR material to be positioned within the crotch of the stapler and cover all cartridge and anvil holes.

### Example 7

The card per example 6 was further modified to include a touch up pad, 2.5″×0.5″×0.04″ (l×w×thickness), made from the same material as the precut SLR.

The following three examples are intended to be comparative examples illustrating the advantages of the present invention against alternative devices. These examples are not necessarily embodiments of the present invention (although they may be) but serve to highlight various important aspects of the invention. For example, comparative Example 8 highlights the concept that a thin plastic application card of the present invention is preferable to a necessarily thicker application device that includes a foam receiving area. Comparative Example 9 highlights the concept that a thin synthetic buttress material fabricated with a uniform adhesive coating as in an embodiment of the present invention has desirable characteristics as compared to an application device such as that described in U.S. Pat. No. 5,752,965, which applies an adhesive gel or the like that requires a foam-type resilient equalization means to establish a uniform adhesion.

### Comparative Example 8

The card per example 5 was modified by adding a 0.2″ rib around the card for ergonomic ease. Addition of the rib made the card less flexible. This change also provided the card with additional stiffness to aid deployment, especially while pivoting the card.

### Comparative Example 9

A card comprised of a foam receiving area (approximately 0.125″ thickness) was constructed and a precut SLR material and a pressure sensitive adhesive was attached to the foam via clips. The SLR was then positioned onto a surgical stapler in the same manner as in Example 4. Upon positioning the SLR into the stapler, the thickness of the foam card caused the SLR material to prematurely touch the stapler causing the SLR material to bunch and not cover the entire staple line.

### Comparative Example 10

A Peri-Strips Dry dispenser (Synovis, Part # 73133-07588, St. Paul Minn.) was modified by replacing the foam pad with a 0.035″ thick polypropylene card. The supplied adhesive (Synovis, Part # 7300-101023, St. Paul Minn.) was then added in a continuous bead to the SLR material, per instructions for use, and the assembly was positioned within the working surface of the stapler jaws. The supplied adhesive was gel-like in nature, and required pressure to evenly distribute the adhesive onto the material. The stapler (Ethicon, Proximate 75, Cincinnati Ohio) was then closed and then re-opened to examine the deployment of the SLR onto the stapler jaws. The SLR material did adhere to the working surface of the stapler at the center of the buttress material where the bead was applied, but the adhesive was not evenly distributed across the entire width of the SLR to adhere the edges of the SLR to the stapler.

The foregoing disclosure of the preferred embodiments of the present invention has been presented for purposes of illustration and description. It is not intended to be exhaustive or to limit the invention to the precise forms disclosed. Many variations and modifications of the embodiments described herein will be apparent to one of ordinary skill in the art in light of the above disclosure. The scope of the invention is to be defined only by the claims appended hereto, and by their equivalents.

Further, in describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

1. An apparatus for applying surgical buttress material to a surgical stapler comprising:
a base member comprising at least a first side and a second side;
a first tensioning member extending outwardly from said first side;
a second tensioning member spaced apart from said first tensioning member and extending outwardly from said first side;
a surgical buttress material disposed over said base member wherein at least a portion of opposing edges of said surgical buttress material is coupled to at least a portion of said first and second tensioning members; and
wherein said surgical buttress material is maintained under tension by said first and second tensioning members prior to application.

2. The apparatus of claim 1 wherein said surgical buttress material further comprises perforations along said opposing edges of said surgical buttress material.

3. The apparatus of claim 1 wherein said surgical buttress material is coupled to said first and second tensioning members by posts extending outwardly from said first and second tensioning members.

4. The apparatus of claim 1 wherein said base member further comprises an alignment mechanism.

5. The apparatus of claim 1 further comprising a handle coupled to the base member.

6. An apparatus for applying surgical buttress material to a surgical stapler comprising:
a flexible, substantially planer base member with a first predetermined width, a first surface and a second surface; and
a surgical buttress material with a second predetermined width, wherein said second predetermined width is less than that of said first predetermined width,
wherein at least a portion of opposing edges of the surgical buttress material are coupled to at least a portion of opposing edges on said second surface of said base member, and
wherein the base member is maintained in a substantially u-shaped configuration by said surgical buttress material prior to application.

7. The apparatus of claim 6 wherein said surgical buttress material further comprises perforations along said opposing edges of said surgical buttress material.

8. The apparatus of claim 6 wherein said surgical buttress material is coupled to said base member by posts extending outwardly from said second surface of said base member.

9. The apparatus of claim 6 wherein said base member further comprises an alignment mechanism.

10. The apparatus of claim 6 further comprising a handle coupled to the base member.

11. An apparatus for applying surgical buttress material to a surgical stapler comprising: a base member comprising a void, an elastic portion and a surgical buttress material wherein said base member is coupled to said surgical buttress material at the elastic portion and wherein said surgical buttress material is disposed across said void and is maintained under tension prior to application.

12. The apparatus of claim 11 wherein the base member comprises said elastic component.

13. The apparatus of claim 11 wherein the surgical buttress material comprises said elastic component.

14. The apparatus of claim 11 wherein said buttress material further comprises perforations within an area of the buttress material disposed across said void.

15. (canceled)

16. The apparatus of claim 11 wherein said base member further comprises an alignment mechanism.

17. The apparatus of claim 11 further comprising a handle coupled to the base member.

18. A method for releasably affixing surgical buttress material to a surgical stapler comprising the steps of:
a) obtaining an apparatus according to claim 2;
b) inserting the apparatus between open stapler arms;
c) orienting the stapler arms so that the surgical buttress material is in position to be applied to the arms of the stapler upon contact there between;
d) closing stapler arms upon the surgical buttress material
e) applying pressure sufficient to tear the surgical buttress material along said perforations
f) opening the stapler arms; and
g) removing the apparatus from contact with the stapler arms.

19. A method for releasably affixing surgical buttress material to a surgical stapler comprising the steps of:
a) obtaining an apparatus according to claim 7;
b) inserting the apparatus between open stapler arms;
c) orienting the stapler arms so that the surgical buttress material is in position to be applied to the arms of the stapler upon contact there between;
d) closing stapler arms upon the surgical buttress material
e) applying pressure sufficient to tear the surgical buttress material along said perforations
f) opening the stapler arms; and
g) removing the apparatus from contact with the stapler arms.

20. (canceled)

21. An apparatus for applying surgical buttress material to a surgical stapler comprising a base member with a first side and a second side, a plurality of tensioning members extending outwardly from said first side, and a coupling mechanism arranged on said tensioning members for coupling a surgical buttress material to said apparatus.

22. The apparatus of claim 21 wherein said base member further comprises an alignment mechanism.

23. The apparatus of claim 21 further comprising a handle coupled to the base member.

24. An apparatus for applying surgical buttress material to a surgical stapler comprising:
a base member comprising at least a first side and a second side;
a first tensioning member extending outwardly from said first side to a first point;
a second tensioning member spaced apart from said first tensioning member and extending outwardly from said first side to a second point;
wherein the distance between said first and second point is greater in an uncoupled configuration than in a coupled configuration.

25. The apparatus of claim 24 wherein at least a portion of opposing edges of a surgical buttress material is coupled to at least a portion of both first and second tensioning members when the apparatus is in a coupled position.

26. The apparatus of claim 24 further comprising at least one hinge coupling said first tensioning member to said base member.

27. A method for releasably affixing surgical buttress material to a surgical stapler comprising the steps of:
a) obtaining an apparatus according to claim 24;
b) coupling a surgical buttress material to said apparatus;
b) inserting the apparatus between open stapler arms;
c) orienting the stapler arms so that the surgical buttress material is in position to be applied to the arms of the stapler upon contact there between;
d) closing stapler arms upon the surgical buttress material
e) applying pressure sufficient to separate the surgical buttress material from the apparatus;
f) opening the stapler arms; and
g) removing the apparatus from contact with the stapler arms.
